(19) United States
(12) Patent Application Publication
(10) Pub. No.: US 2013/0280304 A1
(43) Pub. Date: Oct. 24, 2013

(54) METHOD OF ADMINISTRATION OF ACTIVE AGENTS TO NON-HUMAN MAMMALS

(71) Applicant: Douglas Kramer, Granada Hills, CA (US)

(72) Inventor: Douglas Kramer, Granada Hills, CA (US)

(21) Appl. No.: 13/657,817

(22) Filed: Oct. 22, 2012

Related U.S. Application Data
(60) Provisional application No. 61/636,592, filed on Apr. 20, 2012.

(51) Int. Cl. A61K 9/70; A61K 47/36; A61K 47/12; A61K 47/14; A61K 36/00; A61K 47/24; A61K 47/26; A61K 47/18; A61P 3/02; A61K 45/08; A61K 47/42

(52) U.S. Cl. 424/400; 514/789; 514/777; 514/780; 514/784; 514/778; 514/786; 514/774; 514/788; 514/785; 424/725

(57) ABSTRACT

The present invention provides an improved method of administering a medicinal, dental or nutritional agent to a non-human animal, such as a domesticated dog or cat. The method comprises mixing a liquid composition in a mixer at various speeds and pressures for a period of time; pouring the liquid composition onto a conveyor belt propelled by polymer rollers, wherein the polymer rollers allow the components to advance on the conveyor belt without adhering to the belt; evenly distributing the composition on the belt with the use of a knife; heating the composition in one or more hot-air chambers; obtaining a film comprised of the composition; cutting the film into strips; and then administering one or more of the strips to the non-human animal by placing the strip under the tongue of the animal, i.e., by sub-lingual administration.

### FIELD OF THE INVENTION

The present invention relates to methods for the administration and accurate dosage delivery of medicinal active agents to non-human animals and, in particular, to the administration of one or more active agents to pets, where dosage control is potentially critical.

### BACKGROUND OF THE INVENTION

Successful administration of a prescription, OTC medication or a nutritional supplement can be especially problematic when the user is a relatively inbred domesticated animal such as a dog or a cat. While many dogs and other pets are often of a gentle and cooperative nature, such animals may also often be high strung and difficult to control. Even medicines which have an attractive taste may be spit out by the animal which does not understand the combination of taste and texture, and will reflexively attempt to expel the same. Although such attempt may be only partially successful, it may result in partial disgorgement and thus an unacceptable variation in the effective administration dosage. In the case of larger animals, such as dogs, the animal may react by biting or physically fighting off the administration, for example by throwing its body at the person administering the medication, jumping and counteracting the administration with its paws, and so forth. A need exists for an improved method of administering drugs or nutritional supplements to such animals.

### SUMMARY OF THE INVENTION

The present invention fulfills this need by providing an improved method of administering a medicinal, dental or nutritional agent to a non-human animal, such as a domesticated dog or cat. Thus, certain known potential strategies for administration, such as the use of breath strips applied to the top of the animal's tongue for the purpose of controlling bad breath in pets will not function to achieve accurate administration of medications to pets, and in particular dogs.

In accordance with the invention, thin film delivery of a dosage critical medication is implemented by sublingual administration of the thin film medication under the pet's, i.e. dog's tongue. The inventive method is also implementable on larger animals, such as horses, sheep, cattle and so forth which can be particularly difficult to dose conventionally. While the procedure of implementing a particular placement position in the mouth of the animal might be viewed as disadvantageous, compared to administration by application to the top surface of the tongue, the inventive sublingual administration method gives substantial and unexpected advantages in pets and other non-human animals. In particular, the placement sublingually does not appear to interpreted by the animal as a foreign object in the mouth and does not appear to stimulate a disgorgement response.

As alluded to above thin film technology has been available for human administration as an alternative to the use of traditional tablets, capsules and liquids often associated with prescription, over the counter (OTC) medications and nutritional supplements for human administration. Similar in size, shape and thickness to a postage stamp, thin film strips typically are designed for oral administration, with the user placing the strip on the tongue or under the tongue (sublingual) or along the inside of the cheek (buccal). Depending on the active agent being administered, these drug delivery options allow the medication to bypass first pass metabolism by the liver, thereby making the medication more bioavailable. This may provide added advantages in accordance with the present invention. As the strip dissolves, the drug can enter the blood stream enterically, buccally and sublingually.

Thin film drug delivery uses a dissolving film or oral drug strip to administer the drugs or supplements via absorption in the mouth (buccally or sublingually) and/or via the small intestines (enterically). Typically, a film is prepared using hydrophilic polymers that rapidly dissolves on the tongue or buccal cavity, delivering the drug to the systemic circulation via dissolution when contact with liquid is made.

Formulation of oral drug strips involves accommodating both aesthetic and performance characteristics by selecting and adjusting the proportions of strip-forming polymers, plasticizers, sweetening agents, saliva stimulating agents, flavoring agents, coloring agents, stabilizing and thickening agents and one or more active agents.

The inventive method involves the sublingual application of a controlled dose of a medication in strip form. The strip is made by mixing a liquid composition in a mixer at various speeds and optionally under pressure for a sufficient period of time to achieve uniformity in the mix; pouring the liquid composition onto a polymer sheet conveyor belt which is unrolled from one polymer roll and rolled up onto another, wherein the polymer rollers allow the components to advance on the conveyor belt without adhering to the belt; evenly distributing the composition in a layer on the belt with the use of a blade edge positioned over the conveyor surface and spaced from the conveyor surface; heating the composition in one or more hot-air chambers to dry the layer; obtaining a dried film comprised of the composition by peeling the dried composition film off the polymer sheet conveyor; and cutting the film into strips. Following cutting, the animal is dosed by administering one or more of the strips to the non-human animal by placing the strip under the tongue of the animal, i.e., by sublingual administration. Alternatively, the film-forming material, which may be gel or more preferably pullulan based, may be cast by being sprayed into molds and dried using substantially non-moving or circulating or otherwise moving hot air.

Sublingual administration in non-human animals yields an unexpected low level of drug rejection, insofar as it appears that pets, and in particular dogs, do not appear to recognize the sublingual strip as an object in the mouth and do not attempt to reject the same. While the preferred embodiment of the invention contemplates the use of flavorings, even without the same surprising effectiveness in terms of a low rate of expulsion is achieved.

The composition may be comprised of plasticizers, coloring agents and flavoring agents, emulsifiers, solubilizers and one or more active agents.

Active agents that can be administered in the film strips produced according to the methods of the present invention include, without limitation, medicines, oral hygiene agents, botanical extracts or nutritional supplements.

Suitable plasticizers that can be included in the film strips produced according to the methods of the present invention include, without limitation, carrageenan, xanthan, locust bean or arabica.

Suitable solubilizers that can be included in the film strips produced according to the methods of the present invention include, without limitation, citric acid, tartaric malic acid, modified starches, triacetin and gelatin.

Suitable flavoring agents that can be included in the film strips produced according to the methods of the present invention include, without limitation, acesulfame potassium, sucralose, aspartame and stevia.

A suitable emulsifier that can be implemented in the film strips produced according to the methods of the present invention includes soy lecithin.

The combination of ingredients is placed in the mixer is initially at room temperature, or about 10 to 15 degrees C., and is mixed at a speed of about 7500 to about 12,500 RPM in order to homogenize the composition. The mixture is then heated to a temperature sufficient to form a solution and blended for several minutes until a homogeneous mixture is formed. The plasticizers are present in the composition in order to thicken the composition once it is homogenized. After homogenization, the composition is cooled to a temperature of about 5° C. This is accomplished by injecting CO₂ gas into the mixer. The speed of the mixer then is reduced to about 5000 RPM and the pressure inside the mixture is increased to about 4 bars of pressure. After about 5 minutes, the speed of the mixer is increased to about 8500 RPM while the composition rests for a period of time to allow the temperature of the composition to return to room temperature, or about 10 to 15° C. Once it reaches this temperature, the mixed composition is sprayed from the mixer onto the conveyor belt.

Next one or more hot-air chambers heat the composition to a temperature of about 300 to 355° C., which turns the composition into a film which upon exiting the one or more hot air chambers is deformable and somewhat viscous. The film is then wound around a roller. A non-adhesive substance is applied to the film in order to prevent the film from adhering to itself. The wound film roll then is cut into strips of about ¾ inches long and about ½ inch wide. Each of the strips has a thickness of about 45 μm, which can be varied in thickness over a wide range by methods well known in the prior art.

### BRIEF DESCRIPTION OF THE DRAWINGS

A fuller understanding of the invention can be gained from the following description when read in conjunction with the accompanying drawings in which:

FIG. 1 shows the production method of the film strips according to the embodiments of the present invention;

FIG. 2 is a flow chart of one version of a standard prior art manufacturing method for making film strips used in the pharmaceutical industry;

FIG. 3 is a flow chart of a second version of a standard prior art manufacturing method for making film strips used in the pharmaceutical industry;

FIG. 4 is a flow chart of the innovative manufacturing method of film strips according to the embodiments of the present invention;

FIG. 5 is an illustration of a three-layer film strip manufactured according to the methods of the present invention;

FIG. 6 is a photograph of a matrix cutter used to cut a three-layer film strip according to the embodiments of the present invention;

FIG. 7 is an illustration of a film strip sheet having 50 film strips per sheet according to the embodiments of the present invention; and

FIG. 8 is an illustration of a three-layer film strip and the composition of ingredients contained in each layer according to the embodiments of the present invention.

### DETAILED DESCRIPTION OF THE INVENTION

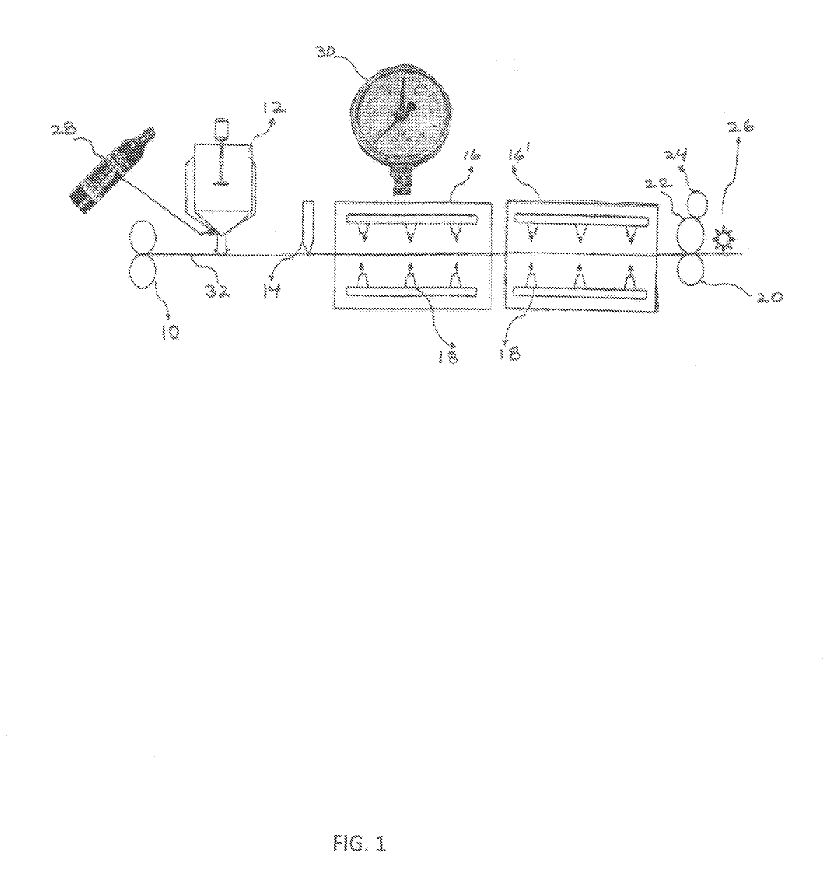

The manufacturing method for making the inventive film strips, which provides enhanced retention of active agents in the composition of the film strips of the present invention as well as enhanced solubility of the film, is shown in FIG. 1. The method comprises the use of a polymer roll 10 which advances a conveyor belt 32 without adhesion of the ingredients of the film strips to the belt 32. A mixer 12 or “jacketed mixer” is used to mix the different components of the matrix of the film strip. The inactive and active ingredients are placed in the mixer 12 in a specific order and mixed at a speed ranging from about 7500 to 12,500 RPM at ambient temperature, i.e., a temperature ranging from about 15 to 20° C. The liquid then is cooled to a temperature of about 5° C., resulting in a very compact, homogeneous, almost solid texture due to a hardening of the consistency of the liquid solution. Once the mixture is homogenized, plasticizer agents and other derivatives, depending on the type of film matrix used, are integrated into the mixture. At the same time, while the thickened mixture is at its lowest temperature and its reactivity is most inhibited, CO₂ gas is injected from a CO₂ canister 28 directly into the mixture. The speed of the mixer 12 then is reduced to about 5000 RPM and the pressure inside the mixer 12 is increased to about 4 bars pressure as read by a pressure gauge 30. At this elevated pressure, the liquefied plasticizers are less efficient, making the other components in the mixture more dissolvable without saturating the consistency of the mixture or its ability to maintain homogeneity of the components therein.

Once the CO₂ gas is dissolved in the mixture and components are completely dissolved, one or more active agents are added to the mixture in a concentration up to four times greater than the saturation point normally observed. More particularly, an average of 20 to 30 mg of active agent per unit of standard film strip. The solution then is rested and the speed of the mixer 12 is increased to 8500 RPM in order to return it to ambient temperature. This prevents crystallization and brittleness upon entering the one or more hot air chambers 16, 16′, which would otherwise result.

After the desired consistency and ambient temperature are reached, a discharge valve of the mixer 12 is opened and the solution is poured onto the conveyor belt 32, which may comprise a polyester film 32 which has been unwound from a roll. The solution then is distributed evenly onto the conveyor belt 32, being spread and thickness limited by a knife 14 (“doctor blade”) which limits the input height of the stream of the solution, ensuring its consistency and equal distribution on the conveyor belt 32 before entering one or more hot air chambers 16, 16′. Excess material removed by knife 14 is collected and distributed again to form additional portions of the desired layer as the properly formed layer is advanced along the production line.

In one embodiment, the solution may be poured into molds.

Hot air nozzles 18 made of PET dry the liquid composition inside the one or more chambers 16, 16′ and form a dried film from the composition.

The temperature inside the one or more hot air chambers 16, 16′ ranges from about 300 to 350° C. The speed of the conveyor belt 32 is coordinated with this temperature in order to ensure proper drying of the film solution. Residence time in the air chambers can vary dependent on ambient conditions.

After this heat cycle is completed and upon exiting the one or more hot air chambers 16, 16′, the aqueous film solution is viscous and strong but still malleable, i.e., texture wrap standard. The dried film then is conveyed by the conveyor belt 32 to top roll 22 which receives a release agent from roll 24. The non-adhesive substance, which acts as a release agent, is applied from a top roll 22 in order to prevent adhesion of the dried film material to itself. After the film is advanced from central roller 20, the film is cut by a perforator 26 into small, thin strips having a length of about ¾ inches and a width of about ½ inch.

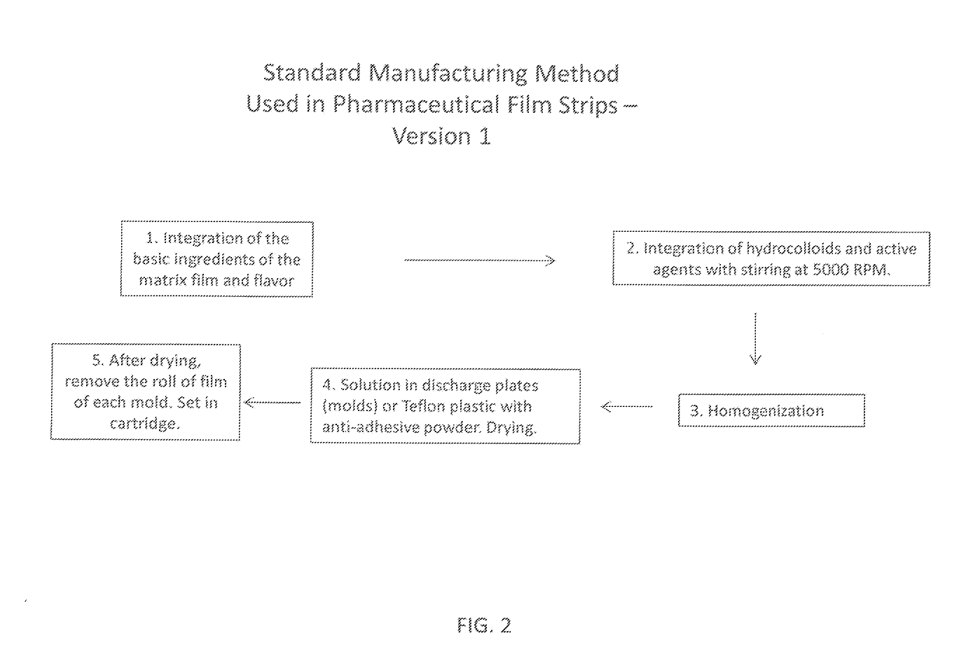

FIG. 2 is a flow chart of one version of a prior art manufacturing method typically used to produce pharmaceutical film strips. Step 1 is integration, that is mixing, of the basic ingredients of the matrix film and flavoring agents. Step 2 is integration of hydrocolloids and active agents with stirring at 5000 RPM. Step 3 is homogenization. Step 4 is pouring the solution onto the conveyor surface (or into discharge plates (molds) or Teflon plastic with anti-adhesive powder) and drying the solution. Step 5 is removing the film from each mold and setting it in a cartridge t type package of conventional design.

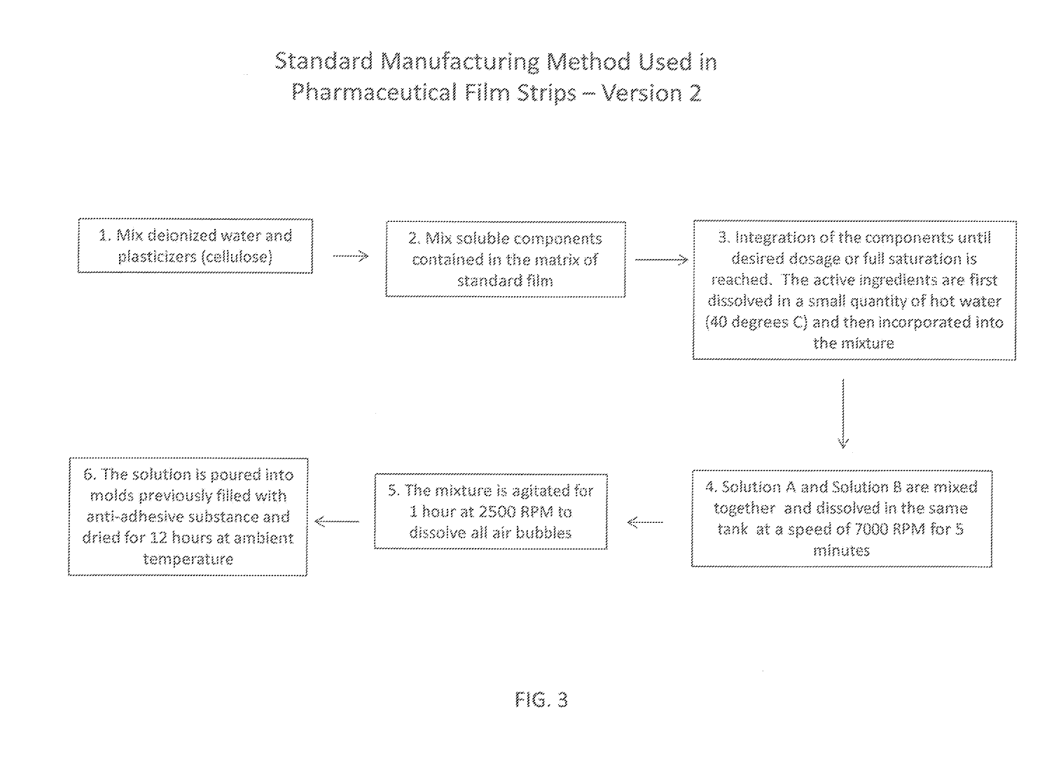

FIG. 3 is a flow chart of a second version of a prior art manufacturing method, also suitable for use with the inventive method, of the type typically used to produce pharmaceutical film strips. Step 1 is mixing deionized water with plasticizers (cellulose) to form solution A. Step 2 is mixing soluble components to be contained in the matrix of the film to form solution B. Step 3 is integration of the components of solution B until the desired dosage or full saturation is reached. The active ingredients are first dissolved in a small quantity of hot water (40° C.) and then incorporated into the mixture. Step 4 is mixing Solution A and Solution B together and dissolving them in the same tank at a speed of about 7000 RPM for about 5 minutes. Step 5 is agitation of the mixture for about one hour at about 2500 RPM to dissolve all air bubbles created. Step 6 is pouring the solution in molds previously filled with anti-adhesive substance and drying for about 12 hours at ambient temperature.

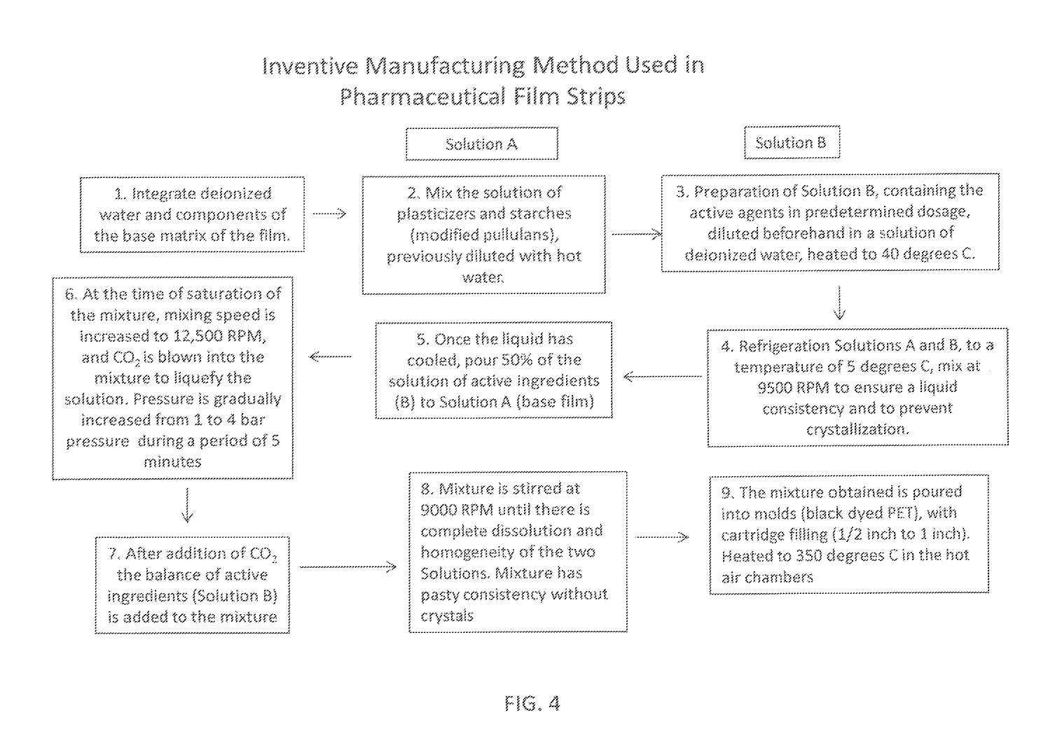

FIG. 4 is a flow chart of the innovative manufacturing method used to produce film strips of the present invention. Step 1 is integration of deionized water and the components of the base matrix of the film. Step 2 is mixing the solution of plasticizers and starches (ie, modified pullulans) Pullulan is an extracellular bacterial polysaccharide produced from starch by Aureobasidium pullulans. It is a linear polysaccharide made up of linked maltotriose residues. As an odorless white colored powder, pullulan is easily soluble in water to make clear and viscous solution. This polymer also has high adhesion, sticking, lubrication, and film forming abilities.

The pullulans have been previously diluted with hot water. Step 3 is preparation of Solution B, containing the active agents in predetermined dosage, diluted beforehand in a solution of deionized water, heated to 40° C. Step 4 is refrigeration of Solutions A and B, to a temperature of 5° C., and then mixing at about 9500 RPM to ensure a liquid consistency and to prevent crystallization. Step 5 is pouring 50% of the solution of active ingredients (B) into Solution A (base film). Step 6 is increasing the speed to about 12,500 at the time of saturation of the mixture, then blowing CO₂ into the mixture to liquefy the solution, and then increasing the pressure from 1 to about 4 bars pressure during a period of about 5 minutes. Step 7 is to add the balance of active ingredients (Solution B) to the mixture. Step 8 is mixing the mixture at about 9000 RPM until there is complete dissolution and homogeneity of the two Solutions. The mixture has pasty consistency without crystals. Step 9 is pouring the mixture obtained into molds, with cartridge filling (½ inch to 1 inch). The mixture then is heated to 350° C. in the hot air chambers.

Traditional One-Layer Manufacturing Method of Film Strips with Added Medicinal Active Ingredients

The standard manufacturing method to produce film strips employed by the pharmaceutical industry (e.g., Breath Strips by Pfizer under the trademark Listerine), has a high dissolvability ratio of 1 g/4 ml of saliva and a thickness of about 30 μm. The composition is homogeneous, which is an essential characteristic for effective film strips, ensuring consistency and sustainability of the film strips.

Three-layer Manufacturing Method of Film Strips with Two Active Ingredients

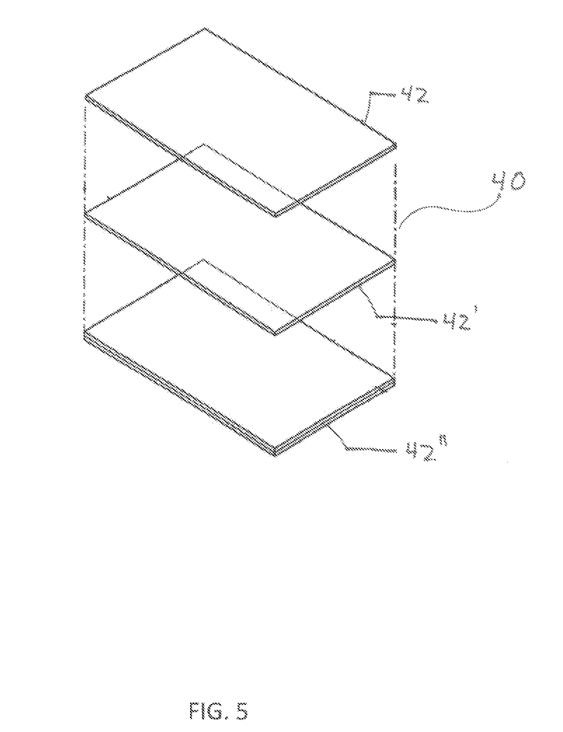

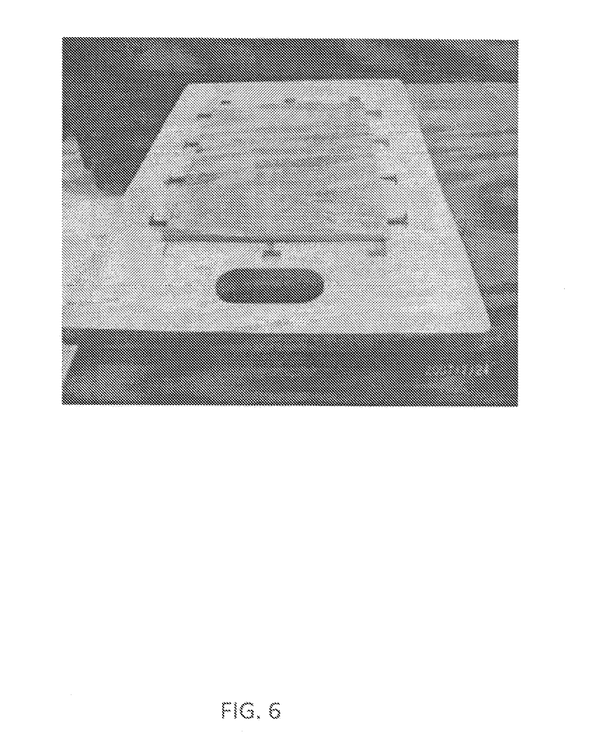

As shown in FIG. 5, the present invention provides a film strip 40 with three layers 42, 42′, 42″ which allows for the integration and even distribution of two active ingredients which typically would be incompatible using traditional production methods of the pharmaceutical industry. FIG. 6 is a photograph of a matrix cutter used to cut a three-layer film strip manufactured according to the methods of the present invention.

Prior art methods of manufacturing film strips provide film strips in units which are ready for consumption. The film strips of the present invention have as many as three layers of film. Optionally, the first of the layers is sprayed onto the conveyor, the second deposited over the first and the third deposited over the second. After formation, the uncut sheet of film, is maintained at room temperature for about 3 to 4 hours depending on the speed of drying. As an alternative to multiple coating, three single layer sheets may be integrated into one unit. This may be done by taking still somewhat tacky, not completely dried sheets (formed for example on a moving polymer resin conveyor belt), assembling them in a multilayer sandwich and adhering them to each other. Assembly may be done in a support which acts as a sort of mold. Once the three sheets are juxtaposed into this unit, the mold containing the three sheets is passed into hot air chambers, or a heat tunnel, which has a temperature of about 350° C., to allow the three layers to fully dry and thus fuse together. After fusion of the three layers, the film strip is cut into unit slices by using a cutting die of the type commonly used in the printing arts.

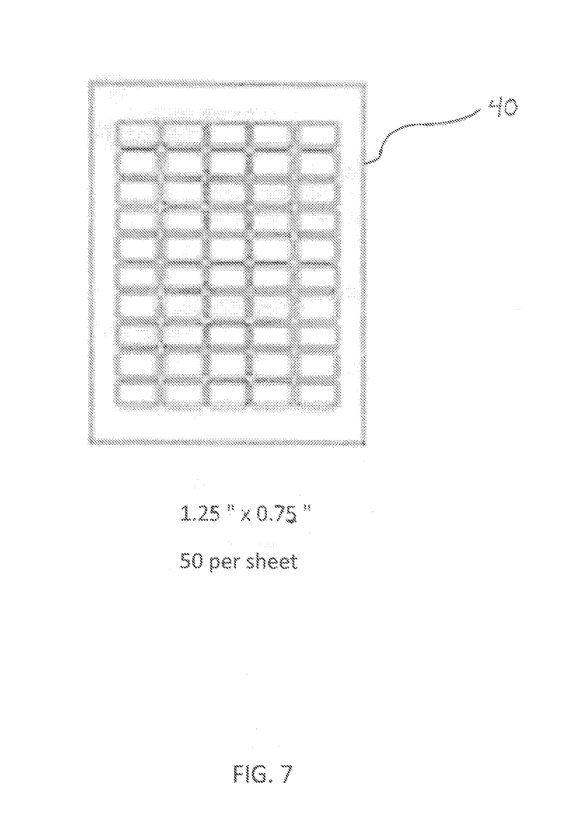

The dimension of the three-layer film strip with multiple coatings is about 1.25″×0.75″, while having a thickness typically 30 μm but not exceeding about 45 μm, larger than a film strips used in human dose administration. This has been determined in accordance with the invention to provide more reliable dosaging. A film strip package 40 having fifty film strips per sheet, manufactured according to the methods of the present invention, is shown in FIG. 7. As discussed above, prior art manufacturing methods of film strips have a ratio of dissolvability of 1 g/4 ml of saliva.

The three-layer film strips of the present invention have the same dissolvability ratio even though the thickness of the film strip is somewhat thicker (45 μm compared to 30 μm for single layer strips), with each layer being about 15 μm and produced separately.

To achieve the same level of dissolvability as the thinner, human dosage film strips, inactive ingredients are incorporated into the base of the film strip medium which does not contain active ingredients, and thus there is an unused area for absorption of the ingredients. Suitable inactive ingredients of the present invention include, without limitation, salivary stimulants such as citric acid, malic acid and ascorbic acid. The combination of these three salivary stimulants compensates for the more difficult dissolvability of the multi-ply film strip by increasing saliva production.

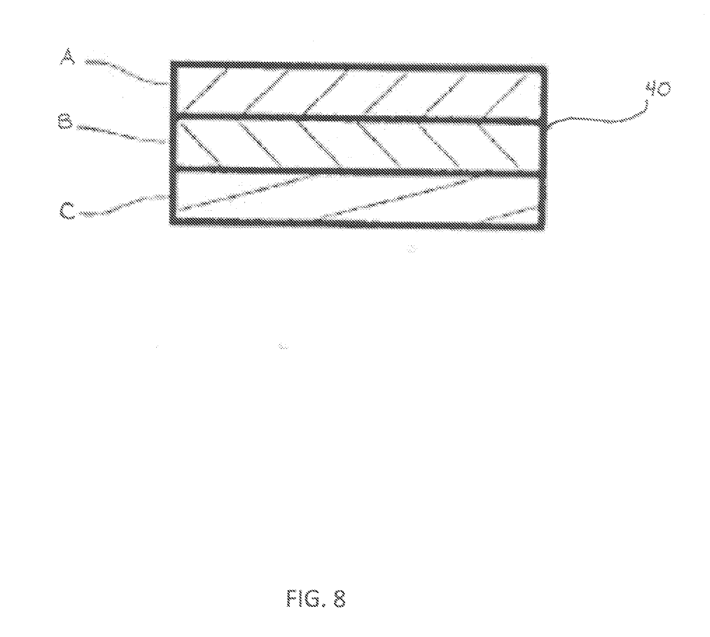

FIG. 8 shows a film strip 40 in cross-section in which the composition of ingredients is homogeneous in each of the three layers of the film strip 40. Two incompatible active ingredients typically present in the same film strip in prior art methods of production are present but separated from one another by the production of a three-layer film strip according to the methods of the present invention. The first layer, A, contains active ingredient A, the second layer, B, contains a film-based adhesive, and the third layer, C, contains active ingredient B. Active ingredient B typically will not be compatible with active ingredient A, and thus is separated as shown in FIG. 8.

The enhanced soluble film strips of the present invention are comprised of three main dietary groups:

(1) Plasticizers such as gums (carrageenan, xanthan, locust bean, arabica) and polymers. The use of gums and polymers (amino branches grouped in different glucose/glycerol molecules) ensure stability, dissolvability and durability of the structure of the film strip.
(2) Coloring agents and flavors such as the sweeteners acesulfame potassium, sucralose, aspartame and stevia, flavors and natural colors and/or artificial colors. Sweeteners are used for their high concentration of sugar contained in a small density and for their dissolvability.
(3) Active agents such as botanical extracts, vitamin and mineral supplements and medicines.

The film strips of the present invention are able to incorporate novel and oftentimes incompatible ingredients due to the innovative methods of the present invention, e.g., heating the solutions, cooling the solutions, anti-emulsifiers, and use of CO₂; all of which together improves the retention capacity of the active ingredients in the film. Thus, the methods of the present invention provide an innovative medicinal or nutritional delivery system which is able to accommodate higher concentrations of ingredients than previously possible, making the application of the technology an effective modality for vetinary administration, and in particular the treatment of dogs. The creation of this innovative medicinal or nutritional delivery system includes, without limitation, the following new components:

(1) Soy Lecithin: used for its emulsifying and lubricating properties, as well as its enhanced ability to distribute active ingredients within the film strips when used at a dosage of about 5 mg per strip or less.
(2) Modified starches: used in place of polysaccharides or other polymers or gums, since a smaller amount of modified starches is needed in the film strip to provide improved containment of the active ingredients. In addition, all ingredients are distributed more homogeneously without clumping, while maintaining a greater solubility in water. Modified starches have never before been used in the production of film strips.
(3) Citric acid and tartaric malic acids: The increased solubility of citric acid and tartaric malic acids allows for their addition to the film strip without increasing the dimensions of the film strip. To achieve the desired dissolution rate for such a film (at least 1 g/6 ml, with 1 g/4 ml saliva being ideal) and to provide efficient and ergonomic absorption for the user, that citric acid, in addition to adding to the complexity of flavor, was found to be the preferred agent for stimulating saliva. Thus, even if a film strip has a higher dissolvability, the time of absorption is the same for the user, due to the greater production of saliva provided by the saliva stimulants.
(4) Triacetin: The index of solubility of the film strip of the present invention also is more effective with the use of triacetin (glycerin triacetate). Triacetin typically is used as an excipient (a carrier vehicle for active ingredients), and to date has never been incorporated into a film matrix, as provided in the present invention.
(5) Gelatin: Gelatin-based animal and vegetable gums in addition to classical gums, due to their dissolution properties at 37° C., allow for their use in larger quantities than classical gums. Gums require standard melt temperatures which are significantly higher than gelatin. As a result, gums cannot exceed film strip thicknesses of 35 to 40 μm. Gelatin, which has a higher solubility index and a lower melting point (37° C.), while having the same properties as xanthan gum or carrageenan (gums typically used as plasticizers to consolidate film strips), allows for the production of thicker film strips, on the order of 40% thicker. In addition, gums leave no residue at a thickness of more than 35 μm. At 37° C., they easily dissolve in the user's mouth.

Provided below are two compositions of film strips used in the methods of the present invention. These make the film strip of the present invention even more soluble while being larger and more durable, and thus allowing incorporation of higher concentrations of active agents, i.e., about 15 to 25 mg of active agents (depending on the concentration desired).

Example 1 gives the preferred formula for the layer holding the active agent/middle adhesive shield layer/salivating agent layer

TABLE 1
Example 1
Composition of Film Strip Base(45 μm)

| Ingredient | % composition |
|---|---|
| Gelatin “bloom high” | 37 |
| tartaric acid | 20 |
| Alginate | 11 |
| Citric acid | 10 |
| UV filtered water | 8 |
| grape flavor | 4.5 |
| sorbitol | 2.7 |
| glycerin | 1.3 |
| Modified starch | 1.33 |
| Carrageenan gum | 1.32 |
| Sorbitan Mono Setarate | 1.3 |
| Sucralose | 0.5 |
| Aspartame | 0.4 |
| Acesulfame Potassium | 0.4 |
| Red dye # 40 0 | 0.15 |
| Blue dye | 0.1 |

Example 2 gives the preferred formula for the layer holding the active agent/middle adhesive shield layer/salivating agent layer

TABLE 2
Example 2
Composition of Film Strip Base (45 μm)

| Ingredient | % (preferred) |
|---|---|
| Gelatin (150 high bloom) | 20.5 |
| Alginate | 19.0 |
| Sorbitol | 15 |
| modified cellulose | 8 |
| UV filtered water | 8 |
| starch modified | 7 |
| Soy lecithin | 7 |
| Menthol | 4 |
| Xylitol | 3.8 |
| Sorbiton Mono Setarate | 3.7 |
| Aspartame | 1.5 |
| Acesulfame Potassium | 1.45 |
| Triacetin | 1 |
| Coloring and mint flavor | 0.05 |

In accordance with the invention, the above ingredients are formulated as described above and the film formed as discussed above.

Example 3 gives the preferred formula for the layer holding the active agent/middle adhesive shield layer/salivating agent layer.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

We claim:

1. A method of administering a medicinal, dental or nutritional agent to a non-human animal, comprising the steps of:
mixing a liquid composition in a mixer at various speeds and pressures for a period of time;
pouring the liquid composition onto a conveyor belt propelled by polymer rollers, said polymer rollers allowing the components to advance on the conveyor belt without adhering to the belt;
evenly distributing the composition on belt with the use of a knife;
heating the composition in one or more hot-air chambers;
obtaining a film comprised of the composition;
cutting the film into strips; and
administering one or more of the strips to a non-human animal by sub-lingual administration.

2. The method according to claim 1, wherein the composition includes plasticizers, coloring agents and flavors, emulsifiers, solubilizers and one or more active agents.

3. The method according to claim 2, wherein the one or more active agents are selected from the group consisting of botanical extracts, nutritional supplements and medicines.

4. The method according to claim 2 wherein the plasticizers are selected from the group consisting of carrageenan, xanthan, locust bean, pullulan and arabica.

5. The method according to claim 2, wherein the solubilizers are selected from the group consisting of citric acid, tartaric malic acid, modified starches, triacetin and gelatin.

6. The method according to claim 2, wherein the emulsifier is soy lecithin.

7. The method according to claim 2, wherein the flavors are sweeteners selected from the group consisting of acesulfame potassium, sucralose, aspartame and stevia.

8. The method according to claim 1, wherein the composition is mixed in a jacketed mixer initially at a speed of about 7500 to about 12,500 RPM in order to homogenize the composition.

9. The method according to claim 8, wherein the placticizers are present to thicken the homogenized composition, and wherein prior to being poured onto the conveyor belt, the composition is cooled to a temperature of about 5 degrees C., carbon dioxide gas is injected into the mixer, the speed of the mixer is reduced to about 5000 RPM, the pressure inside the mixture is increased to about 4 bars pressure, and the speed of the mixer then is increased to about 8500 RPM while the composition rests for a period of time to allow the temperature of the composition to reach about 10 to 15 degrees C.

10. The method according to claim 1, wherein the composition is heated in the one or more hot-air chambers to a temperature of about 300 to 350 degrees C.

11. The method according to claim 1, wherein the composition has a viscous and malleable consistency after exiting the one or more hot-air chambers.

12. The method according to claim 1, wherein the film is wound around a central roller and a non-adhesive substance is applied from a top roll to the film in order to prevent the film from adhering to itself.

13. The method according to claim 1, wherein each of the strips is about ¾ inches long and about ½ inch wide.

14. The method according to claim 1, wherein each of the strips has a thickness of about 45 μm.

15. The method according to claim 1, wherein the non-human animal is a domesticated dog or cat.
